(19) United States
(12) Patent Application Publication
(10) Pub. No.: US 2022/0324826 A1
(43) Pub. Date: Oct. 13, 2022

(54) CANNABICHROMENE COMPOSITIONS AND METHODS OF SYNTHESIZING CANNABICHROMENE

(71) Applicant: Canopy Growth Corporation, Smiths Falls (CA)

(72) Inventors: Daniel BRUMAR, Smiths Falls (CA); Ben GEILING, Smiths Falls (CA); Mohammadmehdl HAGHDOOST MANJILI, Smiths Falls (CA)

(21) Appl. No.: 17/753,609

(22) PCT Filed: Dec. 24, 2020
(86) PCT No.: PCT/CA2020/051799

§ 371 Date: Mar. 9, 2022

Related U.S. Application Data
(60) Provisional application No. 63/046,509, filed on Jun. 30, 2020.
(60) Provisional application No. 62/953,378, filed on Dec. 24, 2019.

(51) Int. Cl. C07D 311/58; C07C 39/08

(52) U.S. Cl. CPC C07D 311/58; C07C 39/08

(57) ABSTRACT

Compositions having enhanced cannabichromene (CBC) and abnormal cannabichromene (CBCab) concentrations are disclosed herein as are methods of synthesizing CBC and CBC_{ab}. Relative to conventional methods, the methods of the present disclosure may: (i) be better suited to large-scale conditions in that they do not require dangerous and/or toxic solvents and/or reagents; (ii) provide product mixtures with enhanced CBC_{ab} concentrations; (iii) provide CBC at higher yield; (iv) provide easier to purify product mixtures comprising CBC; (v) provide product mixtures that comprise unique ratios of CBC_{ab} relative to other cannabinoids; and/or (vi) provide product mixtures with reduced THC concentrations.

### CROSS-REFERENCE TO RELATED APPLICATIONS

This application claims priority to and benefit of U.S. Provisional Patent Application Ser. No. 62/953,378 filed on Dec. 24, 2019 and U.S. Provisional Patent Application Ser. No. 63/046,509 filed on Jun. 30, 2020, each of which is hereby incorporated by reference in its entirety.

### TECHNICAL FIELD

The present disclosure generally relates to methods for synthesizing cannabinoids and to cannabinoid compositions having unique ratios of cannabinoids. In particular, the present disclosure relates to methods for synthesizing cannabichromene (CBC) and to cannabinoid compositions having enhanced CBC concentrations.

### BACKGROUND

Cannabichromene (CBC) is a cannabinoid found in the cannabis sativa plant, and it is quickly gaining consumer and scientific interest. For example, in-vivo studies on rats indicate that CBC has an anti-inflammatory effect equipotent to the non-steroidal anti-inflammatory agent phenylbutazone. CBC is also known to provide a variety of benefits in association with other cannabinoids via the entourage effect. CBC also has utility as a synthon for chemical applications. In spite of this wide-ranging potential, CBC is not currently used at scale. CBC is typically found in low concentrations in cannabis plant material, extracts, distillates and/or the like. At the same time, separating CBC from such compositions can pose significant challenges as CBC is difficult to crystallize and CBC has similar physical characteristics (e.g. solubility, and/or affinity profile) to a number of other cannabinoids.

For similar reasons, CBC can be difficult to separate from reaction mixtures. Conventional synthetic methods for producing CBC typically lack selectivity and lead to significant quantities of other products, such as tetrahydrocannabinol (THC). This can make CBC difficult to obtain in pure form. THC is psychoactive, and compositions containing THC may be subject to regulatory restrictions or prohibitions on transportation or sale. This has the potential to limit the utility of the CBC compositions, because of the difficulty associated with separating the two cannabinoids. This limitation is often compounded by the lack of scalability of conventional methods for synthesizing CBC. At the same time, conventional methods for synthesizing CBC typically rely on toxic solvents and reagents, which have the potential to further limit the utility of the resulting products.

For at least the foregoing reasons, alternative methods for producing CBC are desirable as are cannabinoid compositions with enhanced CBC concentrations.

### SUMMARY

The present disclosure is based on extensive research and development directed at overcoming at least some of the current impediments to advancing the state-of-the-art in cannabichromene (CBC)-related applications. As exemplified by the examples set out herein, the present disclosure advances this field with the provision of alternative methods of synthesizing CBC and abnormal cannabichromene (CBC_{ab}), and derivatives thereof. The present disclosure also provides cannabinoid compositions having unique ratios of CBC_{ab} to other cannabinoids. The methods of the present disclosure may be better suited to industrial scale in that they do not require dangerous and/or toxic solvents and/or reagents. The methods of the present disclosure may provide for more efficient purification of CBC. The methods of the present disclosure may provide access to: (i) CBC in high purity; (ii) mixtures of cannabinoids, including mixtures of CBC and CBC_{ab}, that have unique cannabinoid ratios, and/or (iii) mixtures of cannabinoids with reduced THC concentrations. Importantly, the present disclosure also provides synthetic methods that provide product mixtures with enhanced CBCab concentrations.

Select methods of the present disclosure allow synthesizing CBC and/or CBC_{ab}, and derivatives thereof, from citral and a modified resorcinol. Without being bound to any particular theory, the present disclosure asserts that the ability to synthesize CBC and/or CBC_{ab} as demonstrated herein results from a base-promoted reaction. In particular, the examples of the present disclosure indicate that choice of base and the choice of solvent affect the amount of CBC and CBC_{ab} formed. The methods of the present disclosure may provide access cannabinoid compositions with unique ratios of CBC_{ab} to CBC.

In select embodiments, the present disclosure relates to a method of preparing CBC_{ab} or a CBC_{ab} derivative, the method comprising heating a reaction mixture comprising citral, a modified resorcinol, and a base to form a product mixture, wherein the product mixture comprises at least about 0.5% w/w CBC_{ab}.

In select embodiments, the present disclosure relates to methods of preparing CBC or a CBC derivative, the method comprising heating a reaction mixture comprising citral, a modified resorcinol, a base, and a solvent to form a product mixture, wherein at least one of: the base, and the solvent have low toxic potential.

In select embodiments, the present disclosure relates to a cannabinoid composition comprising at least about 0.5% w/w CBC_{ab} or a CBC_{ab} derivative and at least about 1% w/w CBC or a CBC derivative.

Other aspects and features of the present disclosure will become apparent to those ordinarily skilled in the art upon review of the following description of specific embodiments.

### BRIEF DESCRIPTION OF THE DRAWINGS

FIG. 1 shows a high-performance liquid chromatogram for EXAMPLE 1;

FIG. 2 shows a high-performance liquid chromatogram for EXAMPLE 2;

FIG. 3 shows a high-performance liquid chromatogram for EXAMPLE 3;

FIG. 4A shows a high-performance liquid chromatogram for EXAMPLE 5 with valine as the base;

FIG. 4B shows a high-performance liquid chromatogram for EXAMPLE 5 with Ca(OH)₂ as the base;

FIG. 4C shows a high-performance liquid chromatogram for EXAMPLE 5 with MgO as the base;

FIG. 5A shows a high-performance liquid chromatogram for EXAMPLE 6 with heptane as the solvent; and

FIG. 5B shows a high-performance liquid chromatogram for EXAMPLE 6 with propyl acetate as the solvent.

### DETAILED DESCRIPTION

As noted above, the present disclosure provides alternative methods of synthesizing cannabichromene (CBC) and abnormal cannabichromene (CBC_{ab}), and derivatives thereof.

In the context of the present disclosure, the description relating to properties, features and advantages of the methods and compositions disclosed herein in relation to “CBC_{ab} ” and “CBC” may apply equally to derivatives of these compounds depending on the modified resorcinol used in the synthesis methods. For example, the description herein of CBC_{ab}:CBC ratios may be taken to apply equally to ratios of derivatives of these compounds.

Relative to conventional methods, the methods of the present disclosure may: (i) be better suited to large-scale conditions in that they do not require dangerous and/or toxic solvents and/or reagents; (ii) provide product mixtures with enhanced CBC_{ab} concentrations; (iii) provide CBC at higher yield; (iv) provide easier to purify product mixtures comprising CBC; (v) provide product mixtures that comprise unique ratios of CBC_{ab} relative to other cannabinoids; and/or (vi) provide product mixtures with reduced THC concentrations. The present disclosure asserts that access to CBC via methods disclosed herein may be desirable in both medicinal and recreational contexts. Moreover, the present disclosure asserts that access to CBC via the methods disclosure herein is desirable to synthetic chemists.

The present disclosure provides cannabinoid compositions having unique ratios of cannabinoids, including unique ratios of CBC_{ab} to CBC. Mixtures of cannabinoids may provide enhanced medicinal and/or recreational effects, for example via the entourage effect. The present disclosure provides cannabinoid compositions having reduced THC concentrations which may thereby avoid being subject to regulatory restrictions or prohibitions on transportation or sale. In some embodiments, the methods of the present disclosure provide access to high purity CBC and high purity CBC_{ab}, and derivatives thereof, for example by synthesizing CBC, CBC_{ab} and/or derivatives thereof from citral and a modified resorcinol. High purity CBC and high purity CBC_{ab}, and derivatives thereof, may be employed as an active pharmaceutical ingredient (API) for recreational and/or medicinal formulations, or as a synthon for chemical applications. High purity CBC and high purity CBC_{ab}, and derivatives thereof, may be essentially free of THC and may be obtained from cannabinoid compositions having reduced THC concentrations. In some embodiments, the methods of the present disclosure may provide product mixtures with enhanced CBC_{ab} concentrations, as CBC_{ab} is not found in cannabis plant material.

Without being bound to any particular theory, the present disclosure asserts that the ability to synthesize CBC and/or CBC_{ab} as demonstrated herein results from a reaction between citral, a modified resorcinol, and a base. Non-exclusive examples of bases that react to form CBC and/or CBC_{ab} include inorganic bases (e.g. Ca(OH)₂, MgO, ZrO₂) and amines. In particular, the examples of the present disclosure indicate that choice of base and the choice of solvent affect the amount of CBC and CBC_{ab} formed.

In select embodiments, the present disclosure relates to a method of preparing CBC_{ab} or a CBC_{ab} derivative, the method comprising heating a reaction mixture comprising citral, a modified resorcinol, and a base to form a product mixture, wherein the product mixture comprises at least about 0.5% w/w CBC_{ab} or a CBC_{ab} derivative.

In an embodiment, the product mixture comprises between about 1% w/w CBC_{ab} and about 95% w/w CBC_{ab}, or more particularly between about 1% w/w CBC_{ab} and about 75% w/w CBC_{ab}, between about 1% w/w CBC_{ab} and about 50% w/w CBC_{ab}, or between about 1% w/w CBCab and about 25% w/w CBC_{ab}. In an embodiment, the product mixture comprises between about 2.5% w/w CBC_{ab} and about 50% w/w CBC_{ab}, more particularly between about 25% w/w CBCab and about 40% w/w CBC_{ab}. In an embodiment, the product mixture comprises between about 2.5% w/w CBC_{ab} and about 25% w/w CBC_{ab}. In an embodiment, the product mixture comprises about 0.5%, about 1.0%, about 2.5%, about 5.0%, about 7.5%, about 10.0%, about 12.5%, about 15.0%, about 17.5%, about 20.0%, about 22.5%, about 25%, or greater w/w CBC_{ab}. A product mixture comprising a greater amount of CBC_{ab} may enhance the ease with which the product mixture may be purified. For example, a product mixture comprising about 40% w/w CBC_{ab} or greater than about 40% w/w CBC_{ab} may be more easily purified to yield a CBC_{ab} isolate. As will be appreciated from the disclosure herein, reference to CBC_{ab} in this paragraph equally applies to CBC_{ab} derivatives and the amounts thereof.

In the context of the present disclosure, the term “CBC” refers to cannabichromene or, more generally, to cannabichromene-type cannabinoids. Accordingly the term “CBC” includes: (i) acid forms, such as “A-type”, “B-type”, or “AB-type” acid forms; (ii) salts of such acid forms, such as Na⁺ and/or Ca²⁺ salts of such acid forms; (iii) ester forms, such as those formed by hydroxyl-group esterification to form traditional esters, sulphonate esters, and/or phosphate esters; and/or (iv) various stereoisomers. In select embodiments of the present disclosure, CBC may have the following structural formula:

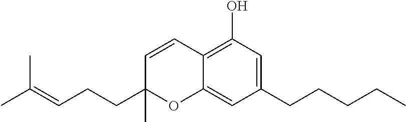 [SMILES: CCCCCc1cc(O)c2c(c1)OC(C)(CCC=C(C)C)C=C2]

In the context of the present disclosure, the term “CBC_{ab}” refers to abnormal cannabichromene or, more generally, to abnormal cannabichromene-type cannabinoids. Accordingly the term “CBC_{ab}” includes: (i) acid forms, such as “A-type”, “B-type”, or “AB-type” acid forms; (ii) salts of such acid forms, such as Na⁺ and/or Ca²⁺ salts of such acid forms; (iii) ester forms, such as those formed by hydroxyl-group esterification to form traditional esters, sulphonate esters, and/or phosphate esters; and/or (iv) various stereoisomers. In select embodiments of the present disclosure, CBC_{ab} may have the following structural formula:

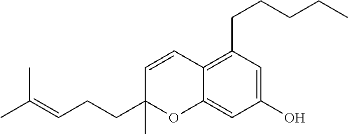 [SMILES: CCCCCc1cc(O)cc2c1C=CC(C)(CCC=C(C)C)O2]

In select embodiments of the present disclosure, citral may have the following structural formula:

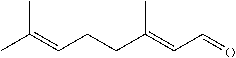

In the context of the present disclosure, the term “resorcinol” refers to resorcinol or, more generally, to resorcinol and modified resorcinols. Resorcinol is a compound of the following structure:

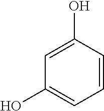

As used in the context of the present disclosure, and in relation to embodiments for the synthesis of CBC and CBC_{ab}, the resorcinol includes the following as a modified resorcinol:

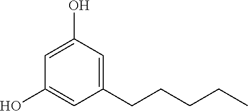

In select embodiments of the present disclosure, the product mixture comprises at least about 0.5% w/w cannabichromene (CBC), more particularly at least about 1% w/w CBC. In an embodiment, the product mixture comprises between about 1% w/w CBC and about 95% w/w CBC, or more particularly between about 1% w/w CBC and about 75% w/w CBC, between about 1% w/w CBC and about 50% w/w CBC, or between about 1% w/w CBC and about 25% w/w CBC. In an embodiment, the product mixture comprises between about 2.5% w/w CBC and about 50% w/w CBC, more particularly between about 5% w/w CBC and about 50% w/w CBC. In an embodiment, the product mixture comprises between about 2.5% w/w CBC and about 30% w/w CBC. In an embodiment, the product mixture comprises about 0.5%, about 1.0%, about 2.5%, about 5.0%, about 7.5%, about 10.0%, about 12.5%, about 15.0%, about 17.5%, about 20.0%, about 22.5%, about 25%, about 27.5%, about 30%, or greater w/w CBC. As will be appreciated from the disclosure herein, reference to CBC in this paragraph equally applies to CBC derivatives and the amounts thereof.

In the context of the present disclosure, the relative quantities of CBC_{ab} and CBC in a particular composition may be expressed as a ratio-CBC_{ab}:CBC. Those skilled in the art will recognize that a variety of analytical methods may be used to determine such ratios, and the protocols required to implement any such method are within the purview of those skilled in the art. By way of non-limiting example, CBC_{ab}:CBC ratios may be determined by diode-array-detector high pressure liquid chromatography, UV-detector high pressure liquid chromatography, nuclear magnetic resonance spectroscopy, mass spectroscopy, flame-ionization gas chromatography, gas chromatograph-mass spectroscopy, or combinations thereof. As will be appreciated from the disclosure herein, reference to CBC_{ab}:CBC ratios may be equally applicable to ratios of derivatives of these compounds, subject to the particular modified resorcinol employed in the methods disclosed herein.

In select embodiments of the present disclosure, a CBC_{ab}:CBC ratio of the product mixture is between about 1000:1 and about 1:1000, between about 500:1 and about 1:500, between about 100:1 and about 1:100, between about 10:1 and about 1:10, between about 5:1 and about 1:5, or between about 2:1 and about 1:2. In an embodiment, the CBC_{ab}:CBC ratio of the product mixture is between about 10:1 and about 1:10. More particularly, in an embodiment, the CBC_{ab}:CBC ratio of the product mixture is between about 4:1 and about 1:3, or more particularly still between about 3:1 and about 1:1.5. In an embodiment, the CBC_{ab}:CBC ratio of the product mixture is about 5:1, about 4:1, about 3:1, about 2:1, about 1:1, about 1:2, about 1:3, about 1:4, or about 1:5. A higher or lower CBC_{ab}:CBC ratio may provide beneficial recreational and/or medicinal effects, or may be of greater benefit to synthetic chemists.

In select embodiments of the present disclosure, the methods relate to the synthesis of a CBC_{ab} derivative of the form:

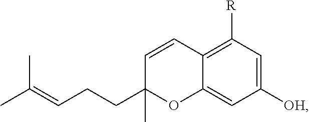

wherein R is methyl, propyl, heptyl, 1,1-dimethylheptyl, phenylethyl, or phenylvinyl. A CBC_{ab} derivative may possess enhanced or modified medicinal and/or recreational effect, and may provide additional functionality for synthetic chemists.

In select embodiments of the present disclosure, the methods relate to the synthesis of a CBC derivative of the form:

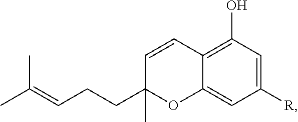

wherein R is methyl, propyl, heptyl, 1,1-dimethylheptyl, phenylethyl, or phenylvinyl. A CBC derivative may possess enhanced or modified medicinal and/or recreational effect, and may provide additional functionality for synthetic chemists.

In select embodiments of the present disclosure, and in relation to the synthesis of the CBC and CBC_{ab} derivatives described herein, the resorcinol may be selected from a modified resorcinol of the form:

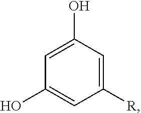

wherein R is methyl, propyl, heptyl, 1,1-dimethylheptyl, phenylethyl, or phenylvinyl. As will be appreciated by those skilled in the art who have benefitted from the teachings of the present disclosure, a derivative of CBC and/or CBC_{ab} synthesized from a modified resorcinol will typically share the same R group.

In select embodiments of the present disclosure, the base comprises an amine. Non-exclusive examples of amines include t-butylamine, pyridine, morpholine, and piperidine. In select embodiments of the present disclosure, the amine comprises a primary amine, a secondary amine, a tertiary amine, or a combination thereof. In select embodiments of the present disclosure, the base comprises a class 3 solvent, a natural amine, or a combination thereof. A non-exclusive example of a base that comprises a class 3 solvent is triethylamine. Class 3 solvents have low toxic potential. Non-exclusive examples of natural amines include amino acids (e.g. valine, lysine). Natural amines typically have low toxic potential, and may be derived from natural sources.

In select embodiments of the present disclosure, the base comprises an inorganic base. In select embodiments of the present disclosure, the base comprises Ca(OH)₂, MgO, ZrO₂, basic Al₂O₃, or a combination thereof. Inorganic bases may have low toxic potential.

In select embodiments of the present disclosure, the methods are performed at a reaction temperature of least about 60° C., more particularly of least about 80° C. In an embodiment, the reaction temperature is between about 80° C. and about 250° C., more particularly between about 80° C. and about 225° C., between about 80° C. and about 120° C., or between about 95° C. and about 110° C. In an embodiment, the reaction temperature is about 80° C., about 85° C., about 90° C., about 95° C., about 100° C., about 105° C., about 110° C., about 115° C., about 120° C., or greater. In an embodiment, the reaction temperature is about 100° C. A higher temperature may increase the rate of reaction, where a higher rate of reaction increases throughput and is thereby more compatible with large scale reactions. A lower temperature may increase the yield of the reaction or reduce the amount of side products formed.

In select embodiments of the present disclosure, the methods are performed for a reaction time of at least about 30 minutes, more particularly at least about 60 minutes. In an embodiment, the reaction time is between about 60 minutes and about 72 hours, more particularly between about 2 hours and about 48 hours, between about 4 hours and about 36 hours, between about 6 hours and about 24 hours, or between about 12 hours and about 24 hours. In an embodiment, the reaction time is between about 18 hours and about 24 hours. In an embodiment, the reaction time is about 30 minutes, about 60 minutes, about 90 minutes, about 2 hours, about 4 hours, about 6 hours, about 8 hours, about 10 hours, about 12 hours, about 18 hours, about 24 hours, about 30 hours, about 36 hours, about 42 hours, about 48 hours, or longer. A shorter reaction time may increase the throughput of the reaction, which increases the rate at which CBC and/or CBC_{ab} is produced. A longer reaction time may increase the yield of the reaction.

In select embodiments of the present disclosure, the methods are performed under a reaction pressure below about 1 bar (below about 100 kPa). In an embodiment, the reaction pressure is between about 0.1 mbar and 1000 mbar, more particularly between about 0.1 mbar and 100 mbar, or between about 0.5 mbar and 10 mbar. Executing the reaction under conditions of reduced pressure may reduce the amount of side products present in a product of the reaction. Non-exclusive examples of side products include cannabicyclol (CBL), tetrahydrocannabinol (THC), cannabicitran (CBT), cannabigerol (CBG), cannabidiol (CBD), and cannabinol (CBN).

In select embodiments of the present disclosure, the method further comprises purifying the product mixture to provide CBC_{ab} or the CBC_{ab} derivative. In an embodiment, the purifying provides CBC_{ab} or a CBC_{ab} derivative with a yield of at least about 5%, more particularly at least about 10%. In an embodiment, the purifying provides CBC_{ab} or a CBC_{ab} derivative with a yield of between about 10% and about 90%, between about 25% and about 75%, or between about 30% and about 50%. A higher yield increases the amount of CBC_{ab} or CBC_{ab} derivative obtained via the methods of the present disclosure. CBC_{ab} or CBC_{ab} derivative with a higher purity may be more readily used as an active pharmaceutical ingredient (API) or chemical synthon.

Non-exclusive examples of purification methods include chromatography, flash chromatography, reversed phase C18 flash chromatography, simulated moving bed chromatography, liquid-liquid extraction, distillation, short-path distillation, and crystallization. Chromatography methods may be surprisingly effective for the isolation of CBC_{ab} or a CBCab derivative from CBC, other cannabinoids, other components of the product mixture, or a combination thereof. In select embodiments of the present disclosure, purifying the product mixture comprises flash chromatography. In select embodiments of the present disclosure, the flash chromatography comprises reversed phase C18 flash chromatography. In select embodiments, purifying the product mixture provides CBC_{ab} or CBC_{ab} derivative with a purity of at least about 95%.

In select embodiments of the present disclosure, the methods comprise contacting the citral, the modified resorcinol, and the base with a class 3 solvent. The class 3 solvent may dissolve at least a portion of the citral, the modified resorcinol, CBC, CBC_{ab}, and/or derivatives thereof, which may increase the rate of reaction. Without being bound to any particular theory, the present disclosure asserts that the properties of the solvent, including the polarity of the solvent, may affect the rate of reaction, the yield of CBC or CBC derivative, the yield of CBC_{ab} or CBC_{ab} derivative, and/or the amount of side products formed.

In select embodiments of the present disclosure, the product mixture comprises less than about 30% w/w tetrahydrocannabinol (THC). In an embodiment, the product mixture comprises less than 30% w/w THC, less than 25% w/w THC, less than 20% w/w THC, less than 15% w/w THC, less than 10% w/w THC, less than 5% w/w THC, less than 2.5% w/w THC, less than 1% w/w THC, less than 0.5% w/w THC, less than 0.3% w/w THC, less than 0.1% w/w THC, or even less. In an embodiment, the product mixture comprises only trace amounts (e.g. less than 0.01% w/w THC). In an embodiment, the product mixture comprises no THC or at least no detectable THC. In the context of the present disclosure, the phrase “less than” includes amounts below the detection limit of appropriate analytical methods.

A lower amount of THC may allow a product mixture to more avoid regulatory restrictions. In select embodiments of the present disclosure, a CBC:THC ratio of the product mixture is between about 10,000:1 and about 1:1, between about 1000:1 and about 1:1, between about 500:1 and about 1:1, between about 100:1 and about 1:1, or between about 10:1 and about 1:1. In an embodiment, the CBC:THC ratio is between about 100:1 and about 10:1, between about 100:1 and about 25:1 between about 100:1 and about 50:1, or between about 100:1 and about 75:1. As will be appreciated from the disclosure herein, reference to CBC in this paragraph equally applies to CBC_{ab}.

In select embodiments of the present disclosure, the product mixture comprises less than about 50% w/w cannabidiol (CBD), less than about 25% w/w CBD, less than about 20% w/w CBD, less than about 15% w/w CBD, less than about 10% w/w CBD, less than about 5% w/w CBD, less than about 1% w/w CBD, less than about 0.3% w/w CBD, less than about 0.2% w/w CBD, or less than about 0.1% w/w CBD.

In select embodiments of the present disclosure, a CBC:CBD ratio of the product mixture may be between about 100:1 and about 1:100, more particularly between about 100:1 and about 1:10. In select embodiments of the present disclosure, the CBC:CBD ratio of the product mixture may be between about 1000:1 and about 900:1, about 900:1 and about 700:1, about 700:1 and about 500:1, about 500:1 and about 300:1, about 100:1 and about 50:1, about 50:1 and about 25:1, about 25:1 and about 10:1, about 10:1 and about 1:1, or about 1:1 and about 1:10. In an embodiment, the CBC:CBD ratio of the product mixture may be about 10:1, about 5:1, about 2:1, about 1:1, about 1:2, about 1:5 or about 1:10. As will be appreciated from the disclosure herein, reference to CBC in this paragraph equally applies to CBC_{ab}.

In select embodiments, the present disclosure relates to a method of preparing cannabichromene (CBC) or a CBC derivative, the method comprising heating a reaction mixture comprising citral, a modified resorcinol, a base, and a solvent to form a product mixture, wherein at least one of: the base, and the solvent have low toxic potential, and wherein the product mixture comprises at least about 0.5% w/w CBC or a CBC derivative, more particularly at least about 1% w/w CBC or a CBC derivative. In the context of the present disclosure, the term “low toxic potential” refers to substances not known as a human health hazard at levels normally accepted in pharmaceuticals.

In an embodiment, the product mixture comprises between about 1% w/w CBC and about 95% w/w CBC, or more particularly between about 1% w/w CBC and about 75% w/w CBC, between about 1% w/w CBC and about 50% w/w CBC, or between about 1% w/w CBC and about 25% w/w CBC. In an embodiment, the product mixture comprises between about 2.5% w/w CBC and about 50% w/w CBC, more particularly between about 5% w/w CBC and about 50% w/w CBC. In an embodiment, the product mixture comprises between about 2.5% w/w CBC and about 30% w/w CBC. In an embodiment, the product mixture comprises about 0.5%, about 1.0%, about 2.5%, about 5.0%, about 7.5%, about 10.0%, about 12.5%, about 15.0%, about 17.5%, about 20.0%, about 22.5%, about 25%, about 27.5%, about 30%, or greater w/w CBC. As will be appreciated from the disclosure herein, reference to CBC in this paragraph equally applies to CBC derivatives and the amounts thereof.

In further embodiments, the product mixture comprises between about 1% w/w CBC and about 70% w/w CBC, between about 10% w/w CBC and about 65% w/w CBC, or between about 40% w/w CBC and about 65% w/w CBC. As will be appreciated from the disclosure herein, reference to CBC in this paragraph equally applies to CBC derivatives and the amounts thereof.

In select embodiments of the present disclosure, the solvent comprises a class 3 solvent. In select embodiments of the present disclosure, the solvent comprises anisole, 2-butanol, heptane, propyl acetate, or a combination thereof.

In select embodiments of the present disclosure, the base comprises an amine. In select embodiments of the present disclosure, the amine comprises a primary amine, a secondary amine, a tertiary amine, or a combination thereof. In select embodiments of the present disclosure, the base comprises a class 3 solvent, a natural amine, or a combination thereof.

In select embodiments of the present disclosure, the base comprises an inorganic base. In select embodiments of the present disclosure, the base comprises Ca(OH)₂, MgO, ZrO₂, basic Al₂O₃, or a combination thereof.

In select embodiments of the present disclosure, the product mixture comprises at least about 0.5% w/w CBC_{ab}. In an embodiment, the product mixture comprises between about 1% w/w CBC_{ab} and about 95% w/w CBC_{ab}, or more particularly between about 1% w/w CBC_{ab} and about 75% w/w CBC_{ab}, between about 1% w/w CBC_{ab} and about 50% w/w CBC_{ab}, or between about 1% w/w CBC_{ab} and about 25% w/w CBC_{ab}. In an embodiment, the product mixture comprises between about 2.5% w/w CBC_{ab} and about 50% w/w CBC_{ab}, more particularly between about 25% w/w CBC_{ab} and about 40% w/w CBC_{ab}. In an embodiment, the product mixture comprises between about 2.5% w/w CBC_{ab} and about 25% w/w CBC_{ab}. In an embodiment, the product mixture comprises about 0.5%, about 1.0%, about 2.5%, about 5.0%, about 7.5%, about 10.0%, about 12.5%, about 15.0%, about 17.5%, about 20.0%, about 22.5%, about 25%, or greater w/w CBC_{ab}. As will be appreciated from the disclosure herein, reference to CBC_{ab} in this paragraph equally applies to CBC_{ab} derivatives and the amounts thereof.

In select embodiments of the present disclosure, a CBC_{ab}:CBC ratio of the product mixture is between about 1000:1 and about 1:1000, between about 500:1 and about 1:500, between about 100:1 and about 1:100, between about 10:1 and about 1:10, between about 5:1 and about 1:5, or between about 2:1 and about 1:2. In an embodiment, the CBC_{ab}:CBC ratio of the product mixture is between about 10:1 and about 1:10. More particularly, in an embodiment, the CBC_{ab}:CBC ratio of the product mixture is between about 4:1 and about 1:3, or more particularly still between about 3:1 and about 1:1.5. In an embodiment, the CBC_{ab}:CBC ratio of the product mixture is about 5:1, about 4:1, about 3:1, about 2:1, about 1:1, about 1:2, about 1:3, about 1:4, or about 1:5. As will be appreciated from the disclosure herein, reference to CBC_{ab}:CBC ratios may be equally applicable to ratios of derivatives of these compounds, subject to the particular modified resorcinol employed in the methods disclosed herein.

In select embodiments of the present disclosure, and in relation to the synthesis of CBC and/or CBC_{ab}, the modified resorcinol is of the form:

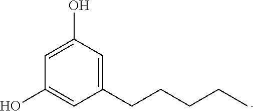

In select embodiments of the present disclosure, and in relation to the synthesis of a CBC derivative and/or a CBC_{ab} derivative, the modified resorcinol is of the form:

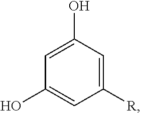

wherein R is methyl, propyl, heptyl, 1,1-dimethylheptyl, phenylethyl, or phenylvinyl.

In select embodiments of the present disclosure, the CBC_{ab} derivative is of the form:

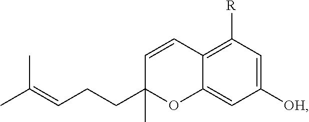

wherein R is methyl, propyl, heptyl, 1,1-dimethylheptyl, phenylethyl, or phenylvinyl.

In select embodiments of the present disclosure, the CBC derivative is of the form:

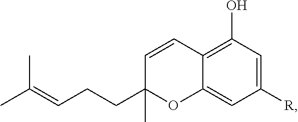

wherein R is methyl, propyl, heptyl, 1,1-dimethylheptyl, phenylethyl, or phenylvinyl.

In select embodiments of the present disclosure, the methods for preparing CBC or a CBC derivative are performed at a reaction temperature of least about 60° C., more particularly of least about 80° C. In an embodiment, the reaction temperature is between about 80° C. and about 250° C., more particularly between about 80° C. and about 225° C., between about 80° C. and about 120° C., or between about 95° C. and about 110° C. In an embodiment, the reaction temperature is about 80° C., about 85° C., about 90° C., about 95° C., about 100° C., about 105° C., about 110° C., about 115° C., about 120° C., or greater. In an embodiment, the reaction temperature is about 100° C.

In select embodiments of the present disclosure, the methods for preparing CBC or a CBC derivative are performed for a reaction time of at least about 30 minutes, more particularly at least about 60 minutes. In an embodiment, the reaction time is between about 60 minutes and about 72 hours, more particularly between about 2 hours and about 48 hours, between about 4 hours and about 36 hours, between about 6 hours and about 24 hours, or between about 12 hours and about 24 hours. In an embodiment, the reaction time is between about 18 hours and about 24 hours. In an embodiment, the reaction time is about 30 minutes, about 60 minutes, about 90 minutes, about 2 hours, about 4 hours, about 6 hours, about 8 hours, about 10 hours, about 12 hours, about 18 hours, about 24 hours, about 30 hours, about 36 hours, about 42 hours, about 48 hours, or longer.

In select embodiments of the present disclosure, the methods for preparing CBC or a CBC derivative are performed under a reaction pressure below about 1 bar (below about 100 kPa). In an embodiment, the reaction pressure is between about 0.1 mbar and 1000 mbar, more particularly between about 0.1 mbar and 100 mbar, or between about 0.5 mbar and 10 mbar.

In select embodiments of the present disclosure, the method further comprises purifying the product mixture to provide CBC or a CBC derivative. In an embodiment, the purifying provides CBC or CBC derivative with a yield of at least about 5%, more particularly at least about 10%. In an embodiment, the purifying provides CBC or a CBC derivative with a yield of between about 10% and about 90%, between about 25% and about 75%, or between about 30% and about 50%. A higher yield increases the amount of CBC or CBC derivative obtained via the methods of the present disclosure. CBC or CBC derivative with a higher purity may be more readily used as an active pharmaceutical ingredient (API) or chemical synthon. Chromatography methods may be surprisingly effective for the isolation of CBC or CBC derivative from CBC_{ab}, other cannabinoids, other components of the product mixture, or a combination thereof. In select embodiments of the present disclosure, purifying the product mixture comprises flash chromatography. In select embodiments of the present disclosure, the flash chromatography comprises reversed phase C18 flash chromatography.

In select embodiments of the present disclosure, the product mixture comprises less than about 30% w/w tetrahydrocannabinol (THC). In an embodiment, the product mixture comprises less than 30% w/w THC, less than 25% w/w THC, less than 20% w/w THC, less than 15% w/w THC, less than 10% w/w THC, less than 5% w/w THC, less than 2.5% w/w THC, less than 1% w/w THC, less than 0.5% w/w THC, less than 0.3% w/w THC, less than 0.1% w/w THC, or even less. In an embodiment, the product mixture comprises only trace amounts (e.g. less than 0.01% w/w THC). In an embodiment, the product mixture comprises no THC or at least no detectable THC. In select embodiments of the present disclosure, a CBC:THC ratio of the product mixture is between about 10,000:1 and about 1:1, between about 1000:1 and about 1:1, between about 500:1 and about 1:1, between about 100:1 and about 1:1, or between about 10:1 and about 1:1. In an embodiment, the CBC:THC ratio is between about 100:1 and about 10:1, between about 100:1 and about 25:1 between about 100:1 and about 50:1, or between about 100:1 and about 75:1. As will be appreciated from the disclosure herein, reference to CBC in this paragraph equally applies to CBCab.

In select embodiments of the present disclosure, the product mixture comprises less than about 50% w/w cannabidiol (CBD), less than about 25% w/w CBD, less than about 20% w/w CBD, less than about 15% w/w CBD, less than about 10% w/w CBD, less than about 5% w/w CBD, less than about 1% w/w CBD, less than about 0.3% w/w CBD, less than about 0.2% w/w CBD, or less than about 0.1% w/w CBD.

In select embodiments of the present disclosure, a CBC:CBD ratio of the product mixture may be between about 100:1 and about 1:100, more particularly between about 100:1 and about 1:10. In select embodiments of the present disclosure, the CBC:CBD ratio of the product mixture may be between about 1000:1 and about 900:1, about 900:1 and about 700:1, about 700:1 and about 500:1, about 500:1 and about 300:1, about 100:1 and about 50:1, about 50:1 and about 25:1, about 25:1 and about 10:1, about 10:1 and about 1:1, or about 1:1 and about 1:10. In an embodiment, the CBC:CBD ratio of the product mixture may be about 10:1, about 5:1, about 2:1, about 1:1, about 1:2, about 1:5 or about 1:10. As will be appreciated from the disclosure herein, reference to CBC in this paragraph equally applies to CBC_{ab}.

In select embodiments, the present disclosure relates to a cannabinoid composition comprising at least about 0.5% w/w abnormal cannabichromene (CBCab) or a CBC_{ab} derivative and at least about 0.5% w/w cannabichromene (CBC) or a CBC derivative.

In select embodiments of the present disclosure, the cannabinoid composition comprises between about 1% w/w CBC_{ab} and about 95% w/w CBC_{ab}, or more particularly between about 1% w/w CBC_{ab} and about 75% w/w CBC_{ab}, between about 1% w/w CBC_{ab} and about 50% w/w CBC_{ab}, or between about 1% w/w CBC_{ab} and about 25% w/w CBC_{ab}. In an embodiment, the cannabinoid composition comprises between about 2.5% w/w CBC_{ab} and about 50% w/w CBC_{ab}, more particularly between about 25% w/w CBC_{ab} and about 40% w/w CBC_{ab}. In an embodiment, the cannabinoid composition comprises between about 2.5% w/w CBC_{ab} and about 25% w/w CBC_{ab}. In an embodiment, the cannabinoid composition comprises about 0.5%, about 1.0%, about 2.5%, about 5.0%, about 7.5%, about 10.0%, about 12.5%, about 15.0%, about 17.5%, about 20.0%, about 22.5%, about 25%, or greater w/w CBC_{ab}. As will be appreciated from the disclosure herein, reference to CBC_{ab} in this paragraph equally applies to CBC_{ab} derivatives and the amounts thereof.

In select embodiments of the present disclosure, the cannabinoid composition comprises between about 1% w/w CBC and about 95% w/w CBC, or more particularly between about 1% w/w CBC and about 75% w/w CBC, between about 1% w/w CBC and about 50% w/w CBC, or between about 1% w/w CBC and about 25% w/w CBC. In an embodiment, the cannabinoid composition comprises between about 2.5% w/w CBC and about 50% w/w CBC, more particularly between about 5% w/w CBC and about 50% w/w CBC. In an embodiment, the cannabinoid composition comprises between about 2.5% w/w CBC and about 30% w/w CBC. In an embodiment, the cannabinoid composition comprises about 0.5%, about 1.0%, about 2.5%, about 5.0%, about 7.5%, about 10.0%, about 12.5%, about 15.0%, about 17.5%, about 20.0%, about 22.5%, about 25%, about 27.5%, about 30%, or greater w/w CBC. As will be appreciated from the disclosure herein, reference to CBC in this paragraph equally applies to CBC derivatives and the amounts thereof.

In select embodiments of the present disclosure, a CBC_{ab}:CBC ratio of the cannabinoid composition is between about 1000:1 and about 1:1000, between about 500:1 and about 1:500, between about 100:1 and about 1:100, between about 10:1 and about 1:10, between about 5:1 and about 1:5, or between about 2:1 and about 1:2. In an embodiment, the CBC_{ab}:CBC ratio of the cannabinoid composition is between about 10:1 and about 1:10. More particularly, in an embodiment, the CBC_{ab}:CBC ratio of the cannabinoid composition is between about 4:1 and about 1:3, or more particularly still between about 3:1 and about 1:1.5. In an embodiment, the CBC_{ab}:CBC ratio of the cannabinoid composition is about 5:1, about 4:1, about 3:1, about 2:1, about 1:1, about 1:2, about 1:3, about 1:4, or about 1:5. As will be appreciated from the disclosure herein, reference to CBC_{ab}:CBC ratios may be equally applicable to ratios of derivatives of these compounds, subject to the particular modified resorcinol employed in the methods disclosed herein.

In select embodiments of the present disclosure, the cannabinoid composition comprises the CBC and CBC_{ab} in the absence of derivative forms thereof.

In select embodiments of the present disclosure, the cannabinoid composition comprises a CBC_{ab} derivative of the form:

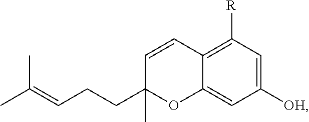

and

a CBC derivative of the form:

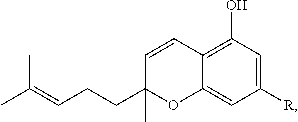

wherein R is a methyl, propyl, heptyl, 1,1-dimethylheptyl, phenylethyl, or phenylvinyl.

In select embodiments of the present disclosure, the cannabinoid composition comprises less than about 30% w/w tetrahydrocannabinol (THC). In an embodiment, the cannabinoid composition comprises less than 30% w/w THC, less than 25% w/w THC, less than 20% w/w THC, less than 15% w/w THC, less than 10% w/w THC, less than 5% w/w THC, less than 2.5% w/w THC, less than 1% w/w THC, less than 0.5% w/w THC, less than 0.3% w/w THC, less than 0.1% w/w THC, or even less. In an embodiment, the cannabinoid composition comprises only trace amounts (e.g. less than 0.01% w/w THC). In an embodiment, the cannabinoid composition comprises no THC or at least no detectable THC.

In select embodiments of the present disclosure, a CBC:THC ratio of the cannabinoid composition is between about 10,000:1 and about 1:1, between about 1000:1 and about 1:1, between about 500:1 and about 1:1, between about 100:1 and about 1:1, or between about 10:1 and about 1:1. In an embodiment, the CBC:THC ratio is between about 100:1 and about 10:1, between about 100:1 and about 25:1 between about 100:1 and about 50:1, or between about 100:1 and about 75:1. As will be appreciated from the disclosure herein, reference to CBC in this paragraph equally applies to CBC_{ab}.

In select embodiments of the present disclosure, the cannabinoid composition comprises less than about 50% w/w cannabidiol (CBD), less than about 25% w/w CBD, less than about 20% w/w CBD, less than about 15% w/w CBD, less than about 10% w/w CBD, less than about 5% w/w CBD, less than about 1% w/w CBD, less than about 0.3% w/w CBD, less than about 0.2% w/w CBD, or less than about 0.1% w/w CBD.

In select embodiments of the present disclosure, a CBC:CBD ratio of the cannabinoid composition may be between about 100:1 and about 1:100, more particularly between about 100:1 and about 1:10. In select embodiments of the present disclosure, the CBC:CBD ratio of the cannabinoid composition may be between about 1000:1 and about 900:1, about 900:1 and about 700:1, about 700:1 and about 500:1, about 500:1 and about 300:1, about 100:1 and about 50:1, about 50:1 and about 25:1, about 25:1 and about 10:1, about 10:1 and about 1:1, or about 1:1 and about 1:10. In an embodiment, the CBC:CBD ratio of the cannabinoid composition may be about 10:1, about 5:1, about 2:1, about 1:1, about 1:2, about 1:5 or about 1:10. As will be appreciated from the disclosure herein, reference to CBC in this paragraph equally applies to CBC_{ab}.

### EXAMPLES

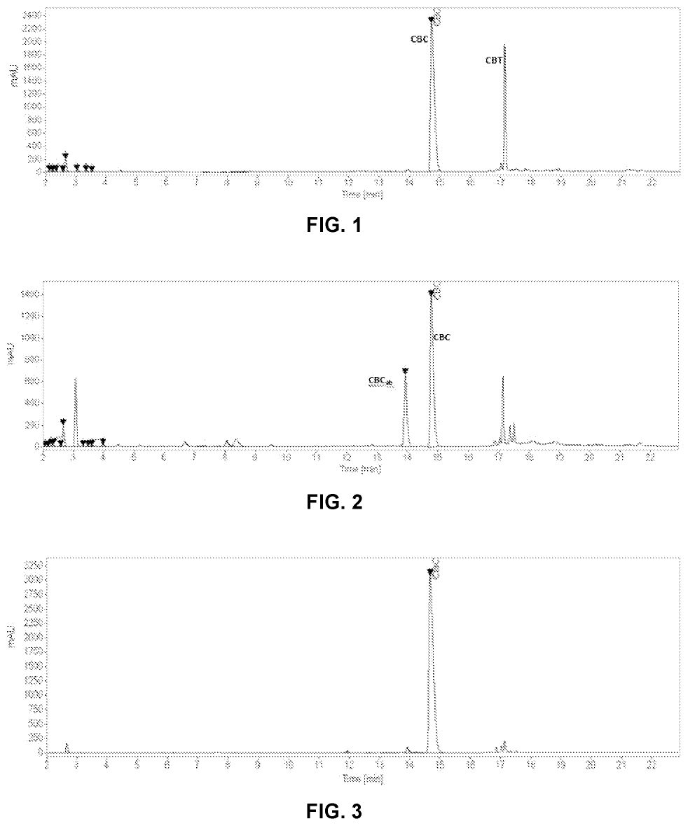

EXAMPLE 1: To a three-necked round bottomed flask, fitted with a dropping funnel and a condenser, under nitrogen atmosphere was added 0.5 g olivetol (2.78 mmol) and 0.2 g (0.36 mL, 2.78 mmol) t-butyl amine in 5 mL toluene and 0.42 g (0.48 mL, 2.78 mmol) of citral was then added dropwise. The mixture was refluxed for 18 hours, after which time it was cooled to room temperature. The reaction mixture was extracted with at least 0.5 MEq of 10% aqueous citric acid solution followed by water. The solution was then dried over anhydrous sodium sulfate, and concentrated by evaporation at reduced pressure to give the crude reaction mixture. The solution was then purified by normal phase silica flash chromatography with a methyl tert-butyl ether/heptane gradient. Analysis of the crude reaction mixture by HPLC (FIG. 1) showed CBC as the major product with a yield in the crude reaction mixture of 61%.

EXAMPLE 2: To a three-necked round bottomed flask, fitted with a dropping funnel and a condenser, under nitrogen atmosphere was added 0.5 g olivetol (2.78 mmol) and 0.28 g (0.39 mL, 2.78 mmol) triethylamine in 5 mL heptane and 0.42 g (0.48 mL, 2.78 mmol) of citral was then added dropwise. The mixture was heated to 110° C. for 18 hours, after which time it was cooled to room temperature. The reaction mixture was extracted with at least 0.5 MEq of 10% aqueous citric acid solution followed by water. The solution was then dried over anhydrous sodium sulfate, and concentrated by evaporation at reduced pressure to give the crude reaction mixture. The solution was then purified by normal phase silica flash chromatography with a methyl tert-butyl ether/heptane gradient. Analysis of the crude reaction mixture by HPLC (FIG. 2) showed CBC as the major product with a yield in the crude reaction mixture of 30%, with significant formation of CBC_{ab}.

EXAMPLE 3: To a three-necked round bottomed flask, fitted with a dropping funnel and a condenser, under nitrogen atmosphere was added 8.0 g olivetol (44.5 mmol) and 3.2 g (5.8 mL, 44.5 mmol) t-butyl amine in 80 mL toluene and 6.72 g (7.68 mL, 44.5 mmol) of citral was then added dropwise. The mixture was refluxed for 18 hours, after which time it was cooled to room temperature. The reaction mixture was extracted with at least 0.5 MEq of 10% aqueous citric acid solution followed by water. The solution was then dried over anhydrous sodium sulfate, and concentrated by evaporation at reduced pressure to give the crude reaction mixture. The solution was then purified by reversed phase C18 flash chromatography with an ethanol/water gradient. Analysis of the crude reaction mixture by HPLC (FIG. 3) showed CBC as the major product with a purified yield in the crude reaction mixture of 28%.

EXAMPLE 4: To a three-necked round bottomed flask, fitted with a dropping funnel and a condenser, under nitrogen atmosphere was added olivetol, 1 MEq t-butyl amine, and 10 mL toluene per g olivetol, and 1 MEq citral was then added dropwise. The mixture was refluxed for 18 hours, after which time it was cooled to room temperature. The reaction mixture was extracted with at least 0.5 MEq of 10% aqueous citric acid solution followed by water. The solution was then dried over anhydrous sodium sulfate, and concentrated by evaporation at reduced pressure to give the crude reaction mixture. The solution was then purified by normal phase silica flash chromatography with a methyl tert-butyl ether/heptane gradient. Analysis by HPLC (not shown) showed CBC as the major product. The amounts of olivetol, reaction time, and yield of CBC in the crude product is shown in TABLE 1. No CBC_{ab} was observed.

TABLE 1
Reaction conditions and crude yield for Example 4.

| olivetol (g) (1 eq.) | time (h) | % CBC (w/w) |
|---|---|---|
| 0.3 | 20 | 62.5 |
| 1 | 23 | 63.5 |
| 12 | 24 | 37.9 |
| 14.5 | 27 | 60 |
| 21 | 20 | 42 |
| 40.8 | 18 | 47.7 |

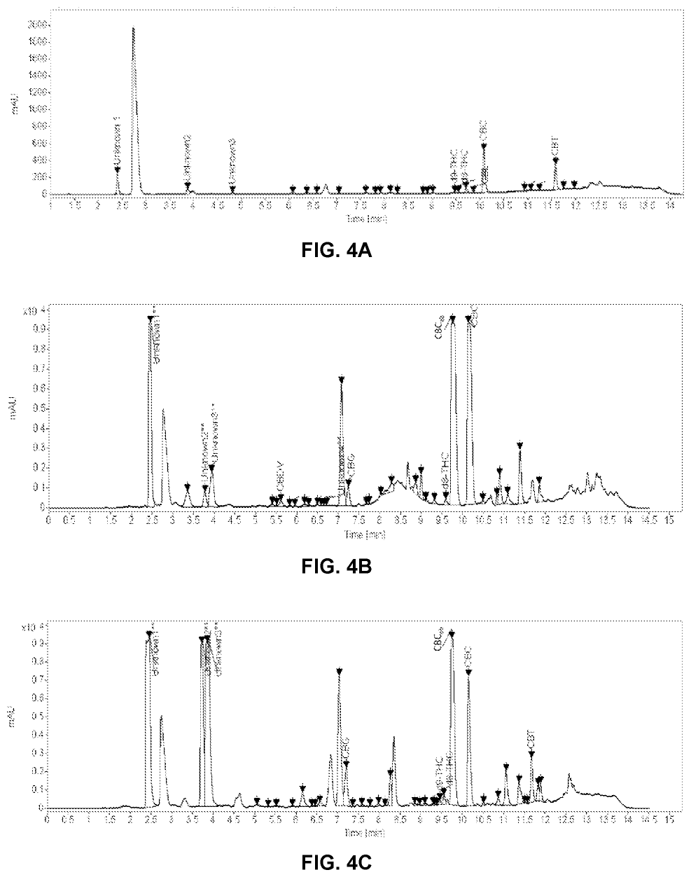

EXAMPLE 5: To a 10 mL tube under nitrogen atmosphere was added 0.3 g olivetol, 3 mL toluene, 1 MEq citral, and base, and the mixture was heated to 100° C. for 18-24 hours, after which time it was cooled to room temperature. If present, solids were removed by filtration, and the reaction mixture was then extracted twice 10% aqueous citric acid solution followed by water. The solution was then dried over anhydrous sodium sulfate, and concentrated by evaporation. Analysis by HPLC (FIG. 4) showed CBC and/or CBC_{ab} as the major product(s). The type and amounts of base, reaction time, and yield of CBC and CBC_{ab} in the crude product is shown in TABLE 2.

TABLE 2
Reaction conditions and crude yield for Example 5.

| | | | time | % CBC | % CBC_{ab} |
|---|---|---|---|---|---|
| Base | Amount of Base | | (h) | (w/w) | (w/w) |
| valine | 1 | MEq | 18 | 27.1 | 0 |
| Ca(OH)₂ | 0.16 | g | 24 | 23.6 | 24.4 |
| MgO | 0.16 | g | 24 | 4.7 | 13 |
| ZrO₂ | 0.16 | g | 24 | 1.3 | 2.9 |
| basic Al₂O₃ | 0.16 | g | 24 | 0.9 | 0.8 |
| lysine | 1 | MEq | 18 | 0 | 0 |

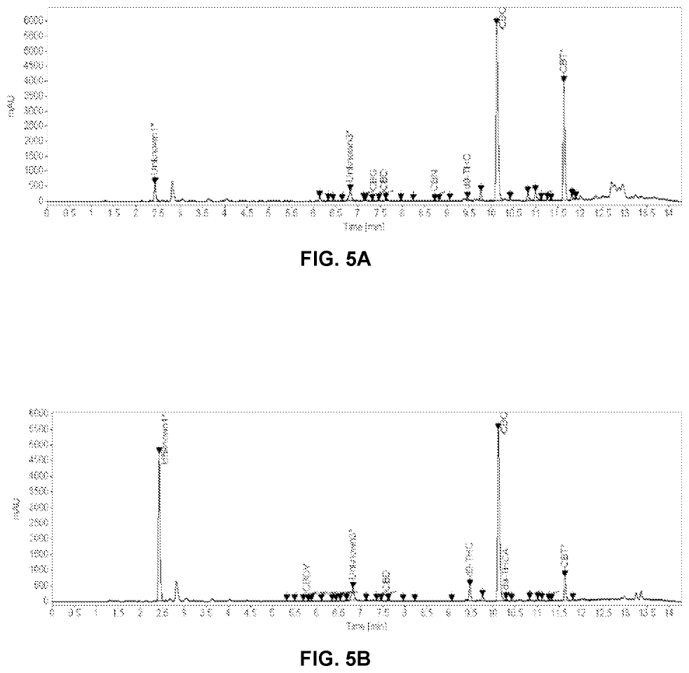

EXAMPLE 6: To a 10 mL tube under nitrogen atmosphere was added 0.3 g olivetol, 3 mL solvent, 1 MEq citral, and 1 MEq t-butyl amine, and the mixture was heated to 100° C. for 18-24 hours, after which time it was cooled to room temperature. If present, solids were removed by filtration, and the reaction mixture was then extracted twice with 10% aqueous citric acid solution followed by water. The solution was then dried over anhydrous sodium sulfate, and concentrated by evaporation. Analysis by HPLC (heptane FIG. 5A and propyl acetate FIG. 5B) showed CBC as the major product. No CBC_{ab} was observed. Some reactions had a drying agent added with the t-butyl amine. The solvent used, reaction time, drying agent used (if any), and yield of CBC in the crude product is shown in TABLE 3.

TABLE 3
Reaction conditions and crude yield for Example 6.

| Solvent | time (h) | Drying Agent | % CBC (w/w) |
|---|---|---|---|
| p-Cymene | 24 | N/A | 64 |
| Anisole | 20 | N/A | 25 |
| heptane | 23 | N/A | 26 |
| 2-butanol | 20 | N/A | 14 |
| propyl acetate | 24 | N/A | 40 |
| propyl acetate | 24 | 3Å Molecular | 43 |
| | | Sieves | |
| propyl acetate | 24 | Anhydrous MgSO4 | 45 |

EXAMPLE 7: To a 15 mL tube was added 0.5 mL propyl acetate, 290 pL citral (1.0 MEq), and 330 pL piperidine (2.2 MEq). The mixture was stirred under nitrogen at room temperature for 2 h. Acetic anhydride (320 pL, 2.2 MEq) was added and the mixture was stirred at 100° C. for 1 h. A solution of olivetol (300 mg, 1.0 MEq) in 2.5 mL propyl acetate was added and the mixture was stirred at 100° C. under nitrogen for 24 h. The reaction mixture was cooled to room temperature, diluted with heptane, washed twice with water, and concentrated by evaporation. Analysis by HPLC showed 2.1% w/w CBC.

EXMAPLE 8: To a 15 mL tube was added 0.5 mL propyl acetate, 290 pL citral (1.0 MEq), and 290 pL morpholine (2.2 MEq). The mixture was stirred under nitrogen at room temperature for 2 h. Acetic anhydride (320 pL, 2.2 MEq) was added and the mixture was stirred at 100° C. for 1 h. A solution of olivetol (300 mg, 1.0 MEq) in 2.5 mL propyl acetate was added and the mixture stirred at 100° C. under nitrogen for 24 h. The reaction mixture was cooled to room temperature, diluted with heptane, washed twice with water, and concentrated by evaporation. Analysis by HPLC showed 6.1% w/w CBC.

EXAMPLE 9: To a mixture of 300 mg olivetol (1.0 MEq) and 290 pL citral (1.0 MEq) under nitrogen was added 3.3 mL (1 MEq) of 0.5 M anhydrous ammonia in dioxane solution at room temperature. The mixture was stirred at 100° C. under nitrogen for 24 h. The reaction mixture was cooled to room temperature, quenched with 10% aqueous citric acid, and diluted with heptane and water. The heptane layer was washed twice with water and concentrated by evaporation. Analysis by HPLC showed 26.8% CBC as the only major product.

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “about” refers to an approximately +/−10% variation from a given value. It is to be understood that such a variation is always included in any given value provided herein, whether or not it is specifically referred to.

It should be understood that the compositions and methods are described in terms of “comprising,” “containing,” or “including” various components or steps, the compositions and methods can also “consist essentially of” or “consist of” the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Although individual embodiments are discussed, the disclosure covers all combinations of all those embodiments. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure. If there is any conflict in the usages of a word or term in this specification and one or more patent(s) or other documents that may be incorporated herein by reference, the definitions that are consistent with this specification should be adopted.

Many obvious variations of the embodiments set out herein will suggest themselves to those skilled in the art in light of the present disclosure. Such obvious variations are within the full intended scope of the appended claims.

1.-71. (canceled)

72. A method of preparing cannabichromene (CBC) or a CBC derivative, the method comprising heating a reaction mixture comprising citral, a modified resorcinol, a base, and a class 3 solvent.

73. The method of claim 72, wherein the modified resorcinol is of the form:

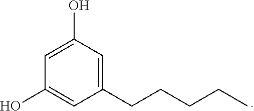

74. The method of claim 72, wherein the modified resorcinol is of the form: 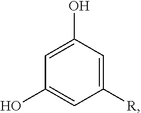
wherein R is methyl, propyl, heptyl, 1,1-dimethylheptyl, phenylethyl, or phenylvinyl.

75. The method of claim 72, wherein the base comprises an amine.

76. The method of claim 75, wherein the amine comprises a primary amine, a secondary amine, a tertiary amine, or a combination thereof.

77. The method of claim 72, wherein the base comprises an inorganic base.

78. The method of claim 77, wherein the base comprises Ca(OH)₂, MgO, ZrO₂, basic Al₂O₃, or a combination thereof.

79. The method of claim 72, wherein the heating is at a reaction temperature of at least 80° C.

80. The method of claim 72, wherein the reaction temperature is between about 80° C. and about 120° C.

81. The method of claim 72, wherein the reaction temperature is between about 95° C. and about 110° C.

82. The method of claim 72, wherein the heating is for a reaction time of at least about 60 minutes.

83. The method of claim 72, wherein the reaction time is between about 60 minutes and about 72 hours.

84. The method of claim 72, wherein the reaction time is between about 12 hours and about 24 hours.

85. The method of claim 72, further comprising purifying a product mixture by flash chromatography.

86. A method of preparing cannabichromene (CBC) or a CBC derivative, the method comprising heating a reaction mixture comprising citral, a modified resorcinol, a base, and a Class 3 solvent at a reaction temperature of between about 80° C. and about 120° C. for at least about 60 minutes.

87. The method of claim 86, wherein the modified resorcinol is of the form:

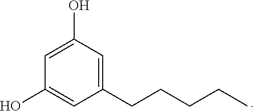

88. The method of claim 86, wherein the modified resorcinol is of the form: 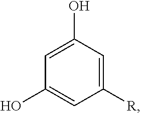
wherein R is methyl, propyl, heptyl, 1,1-dimethylheptyl, phenylethyl, or phenylvinyl.

89. The method of claim 86, wherein the base comprises an amine.

90. The method of claim 89, wherein the amine comprises a primary amine, a secondary amine, a tertiary amine, or a combination thereof.

91. A method of preparing cannabichromene (CBC) or a CBC derivative, the method comprising heating a reaction mixture comprising citral, a modified resorcinol, a base, and a solvent, wherein the solvent is p-cymene.
